(19) United States
(12) Patent Application Publication
(10) Pub. No.: US 2018/0127819 A1
(43) Pub. Date: May 10, 2018

(54) Enhancing Sequencing Performance in Sequencing-by-Synthesis

(71) Applicant: QIAGEN Waltham, Inc., Waltham, MA (US)

(72) Inventors: Luisa Andruzzi, Concord, MA (US); Ben McNally, Medford, MA (US); Angela DeLucia, Cambridge, MA (US); Dona Hevroni, Lexington, MA (US)

(21) Appl. No.: 15/799,241

(22) Filed: Oct. 31, 2017

Related U.S. Application Data
(60) Provisional application No. 62/419,713, filed on Nov. 9, 2016.

(51) Int. Cl. C12Q 1/6874; C12Q 1/6816; C12Q 1/6853

(52) U.S. Cl. CPC C12Q 1/6874; C12Q 2600/16; C12Q 1/6853; C12Q 1/6816

(57) ABSTRACT

The invention relates to methods, compositions, devices, systems and kits as described including, without limitation, reagents and mixtures for determining the identity of nucleic acids in nucleotide sequences using, for example, sequencing by synthesis (SBS) methods. In particular, the present invention contemplates the use of chelators in washing reagents to improve SBS performance.

### FIELD OF THE INVENTION

The invention relates to methods, compositions, devices, systems and kits as described including, without limitation, reagents and mixtures for determining the identity of nucleic acids in nucleotide sequences using, for example, sequencing by synthesis (SBS) methods. In particular, the present invention contemplates the use of chelators in washing reagents to improve SBS performance.

### BACKGROUND

Over the past 25 years, the amount of DNA sequence information that has been generated and deposited into Genbank has grown exponentially. Traditional sequencing methods (e.g., for example Sanger sequencing) are being replaced by next-generation sequencing technologies that use a form of sequencing by synthesis (SBS), wherein specially designed nucleotides and DNA polymerases are used to read the sequence of chip-bound, single-stranded DNA templates in a controlled manner. To attain high throughput, many millions of such template spots are arrayed across a sequencing chip and their sequence is independently read out and recorded.

Systems for using arrays for DNA sequencing are known (e.g., Ju et al., U.S. Pat. No. 6,664,079, herein incorporated by reference). However, there is a continued need for methods and compositions for increasing the efficiency and/or performance for sequencing nucleic acid sequences with automated sequencing.

### SUMMARY OF THE INVENTION

The invention relates to methods, compositions, devices, systems and kits as described including, without limitation, reagents and mixtures for determining the identity of nucleic acids in nucleotide sequences using, for example, sequencing by synthesis (SBS) methods. In particular, the present invention contemplates the use of chelators in washing reagents to improve SBS performance.

In one embodiment, the present invention contemplates a method comprising a chelator compound. In one embodiment, the chelator compound includes, but not limited to, ethylenediaminetetraacetic acid (EDTA) and/or ethylene glycol-bis(β-aminoethyl ether)-N,N,N′,N′-tetraacetic acid (EGTA). In one embodiment, the method comprises a post-cleave wash step with a chelator performed during a sequencing by synthesis (SBS) protocol to improve sequencing performance. In some embodiments, the improved sequencing performance includes, but is not limited to, lower raw error rates and longer read lengths. Although it is not necessary to understand the mechanism of an invention it is believed that such improved SBS protocols may be due to scavenging a cleave reagent compound (e.g., TCEP) that is retained in the flow (e. g., the TCEP is bound to iron-containing surfaces such as iron silicates, ferrotitanium, magnetic head ferrite). Although it is not necessary to understand the mechanism of an invention, it is believed that such cleave reagent residual contamination may build up in the flow cell via surface adsorption mechanisms leading to carry over into the subsequent extension step thus causing premature de-protection of the 3′-OH moiety and impairing single base incorporation rate. Although it is not necessary to understand the mechanism of an invention, it is believed that the chelator may be displacing the contaminant from the surface.

In one embodiment, the present invention contemplates a method of incorporating labeled nucleotides, comprising: a) providing; i) a plurality of nucleic acid primers and template molecules, ii) a polymerase, iii) a washing reagent comprising a chelator, and iv) a plurality of nucleotide analogues wherein at least a portion of said nucleotide analogues is labeled with a label attached through a cleavable linker to the base; b) hybridizing at least a portion of said primers to at least a portion of said template molecules so as to create hybridized primers; c) incorporating a first labeled nucleotide analogue with said polymerase into at least a portion of said hybridized primers so as to create extended primers comprising an incorporated labeled nucleotide analogue; d) cleaving said extended primers comprising an incorporated labeled nucleotide analogue with a solution comprising a cleave reagent; and e) washing said incorporated labeled nucleotide analogue with said washing reagent. In one embodiment, said cleave reagent is selected from the group consisting of tris(2-carboxyethyl)phosphine and tris(hydroxymethyl)-aminomethane HCl. In one embodiment, said chelator binds to said at least one cleave reagent compound. In one embodiment, said SBS instrumentation component comprises an iron-containing surface. In one embodiment, said iron-containing surface is selected from the group consisting of an iron silicate surface, a ferrotitanium surface and a magnetic bead ferrite surface. In one embodiment, said chelator is selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt. In one embodiment, said method further comprises step (e) incorporating a second nucleotide analogue with said polymerase into at least a portion of said extended primers. In one embodiment, said second nucleotide analogue is incorporated into said at least a portion of said extended primer with a reduced error rate as compared to the error rate with a wash reagent without a chelator. In one embodiment, said method further comprises step (e) incorporating an additional nucleotide analogue with said polymerase into said at least a portion of said extended primers. In one embodiment, said additional nucleotide analogue incorporated during a plurality of SBS cycles. In one embodiment, the plurality of SBS cycles ranges between approximately 50-150 cycles, preferably greater than 85 cycles, more preferably greater than 90 cycles, and most preferable greater than 100 cycles. In one embodiment, said at least a portion of said extended primer comprises a longer read length as compared to a baseline buffer wash reagent without a chelator. In one embodiment, said label is fluorescent.

In one embodiment, the present invention contemplates a washing reagent comprising at least one detergent, at least one chelator and a buffer. In one embodiment, the at least one chelator comprises a compound selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt. In one embodiment, the at least detergent is a polysorbate. In one embodiment, the polysorbate is polysorbate 20. In one embodiment, the buffer is a TRIS buffer. In one embodiment, the buffer is a HEPES buffer.

In one embodiment, the present invention contemplates a kit, comprising: i) a first container comprising a washing reagent comprising at least one detergent, at least one chelator and a buffer; and ii) a second container comprising a plurality of nucleotide analogues wherein at least a portion of said nucleotide analogues is labeled with a label attached through a cleavable linker to the base. In one embodiment, the at least one chelator comprises a compound selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt. In one embodiment, the at least detergent is a polysorbate. In one embodiment, the polysorbate is polysorbate 20. In one embodiment, the buffer is a TRIS buffer. In one embodiment, the buffer is a HEPES buffer.

In one embodiment, the present invention contemplates a system comprising a solution of primers hybridized to a template comprising a plurality of nucleotide analogues attached to a cleavable label and a washing reagent comprising at least one detergent, at least one chelator and a buffer. In one embodiment, the hybridized primers and said template are immobilized. In one embodiment, the hybridized primers and said template are in a flow cell. In one embodiment, the at least one chelation compound comprises compounds selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt. In one embodiment, the at least one detergent is a polysorbate. In one embodiment, the polysorbate is polysorbate 20. In one embodiment, the buffer is a TRIS buffer. In one embodiment, the buffer is a HEPES buffer.

A mixture comprising a solution of primers hybridized to a template comprising a plurality of nucleotide analogues attached to a cleavable label and a washing reagent comprising at least one chelator and a buffer. In one embodiment, the hybridized primers and said template are immobilized. In one embodiment, the hybridized primers and said template are in a flow cell. In one embodiment, the at least one chelator comprises compounds selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt. In one embodiment, the reagent further comprises at least one detergent. In one embodiment, the at least one detergent is polysorbate. In one embodiment, the polysorbate is polysorbate 20. In one embodiment, the buffer is a TRIS buffer. In one embodiment, the buffer is a HEPES buffer.

In one embodiment, the present invention contemplates a washing reagent comprising: i) a TRIS HCl buffer; ii) polysorbate 20; and iii) ethylenediaminetetraacetic acid ranging in concentration between approximately 25-50 mM.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” as used herein, in the context of any of any assay measurements refers to +/−5% of a given measurement.

The term “washing reagent” as used herein, refers to a mixture of compounds that are capable of removing and/or reducing the concentration of other reagents used in the performance of an SBS protocol. One particular washing reagent compound comprises a chelation compound including, but not limited to EDTA and/or EGTA.

The term “chelator” and “chelation compound” as used herein refers to a compound that binds to (e.g., for example attaches by either specific or non-specific mechanisms) and/or removes another compound used in one of several SBS reagents. Such a chelation compound may remove, displace and/or reduce the concentration of an SBS reagent compound within a solution, or remove and/or inactivate an SBS reagent compound that is an SBS instrumentation platform contaminant. For example, one such, SBS reagent compound includes, but is not limited to, TCEP. Chelation compounds may include, but are not limited to, EDTA, EGTA, iminodisuccinic acid, polyaspartic acid, ethylenediamine-N,N′-disuccinic acid, methylglycinediacetic acid, and/or L-glutamic acid N,N-diacetic acid, tetrasodium salt.

The term “buffer” as used herein, refers to a mixture of basic salts and a hydrogen exchange compound (either a weak acid or a weak base) that can maintain a stable pH level over a wide range of environmental conditions (e.g., temperature, salinity), including changes in hydrogen ion concentration. For example, such buffers may include, but are not limited to 4-(2-hydroxyethyl)-1-piperazineethanesulfonic acid (HEPES) buffer and/or tris(hydroxymethyl)-aminomethane (TRIS) buffer.

The term “linker” as used herein, refers to any molecule (or collection of molecules) capable of attaching a label and/or chemical moiety that is susceptible to cleavage. In one embodiment, cleavage of the linker may produce toxic radical products. For example, a linker may include, but is not limited to, a disulfide linker and/or are azide linker.

The term “attached” as used herein, refers to any interaction between a first molecule (e.g., for example, a nucleic acid) and a second molecule (e.g., for example, a label molecule). Attachment may be reversible or irreversible. Such attachment includes, but is not limited to, covalent bonding, ionic bonding, Van der Waals forces or friction, and the like.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand. Such nucleic acids may include, but are not limited to, cDNA, mRNA or other nucleic acid sequences.

The term “an isolated nucleic acid”, as used herein, refers to any nucleic acid molecule that has been removed from its natural state (e.g., removed from a cell and is, in a preferred embodiment, free of other genomic nucleic acid).

In some embodiments, the present invention contemplates hybridizing nucleic acid together. This requires some degree of complementarity. As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

Low stringency conditions comprise conditions equivalent to binding or hybridization at 42° C. in a solution consisting of 5×SSPE (43.8 NaCl, 6.9 g/l NaH2PO4.H2O and 1.85 g/l EDTA, pH adjusted to 7.4 with NaOH), 0.1% SDS, 5× Denhardt's reagent {50× Denhardt's contains per 500 ml: 5 g Ficoll (Type 400, Pharmacia), 5 g BSA (Fraction V; Sigma)} and 100 μg/ml denatured salmon sperm DNA followed by washing in a solution comprising 5×SSPE, 0.1% SDS at 42° C. when a probe of about 500 nucleotides in length. is employed. Numerous equivalent conditions may also be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions. In addition, conditions which promote hybridization under conditions of high stringency (e.g., increasing the temperature of the hybridization and/or wash steps, the use of formamide in the hybridization solution, etc.) may also be used.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C0 t or R0 t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tm” is used in reference to the “melting temperature.” The melting temperature is the temperature at which a population of double-stranded nucleic acid molecules becomes half dissociated into single strands. As indicated by standard references, a simple estimate of the Tm value may be calculated by the equation: Tm=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tm.

As used herein the term “stringency” is used in reference to the conditions of temperature, ionic strength, and the presence of other compounds such as organic solvents, under which nucleic acid hybridizations are conducted. “Stringency” typically occurs in a range from about Tm to about 20° C. to 25° C. below Tm. A “stringent hybridization” can be used to identify or detect identical polynucleotide sequences or to identify or detect similar or related polynucleotide sequences. For example, when fragments are employed in hybridization reactions under stringent conditions the hybridization of fragments which contain unique sequences (i.e., regions which are either non-homologous to or which contain less than about 50% homology or complementarity) are favored. Alternatively, when conditions of “weak” or “low” stringency are used hybridization may occur with nucleic acids that are derived from organisms that are genetically diverse (i.e., for example, the frequency of complementary sequences is usually low between such organisms).

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” or (more simply) “template” refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y.

As used herein, the term “polymerase chain reaction” (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the tenth “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “label” or “detectable label” are used herein, to refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference).

In a preferred embodiment, the label is typically fluorescent and is linked to the base of the nucleotide. For cytosine and thymine, the attachment is usually to the 5-position. For the other bases, a deaza derivative is created and the label is linked to a 7-position of deaza-adenine or deaza-guanine.

The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The term “luminescence” and/or “fluorescence”, as used herein, refers to any process of emitting electromagnetic radiation (light) from an object, chemical and/or compound. Luminescence and/or fluorescence results from a system which is “relaxing” from an excited state to a lower state with a corresponding release of energy in the form of a photon. These states can be electronic, vibronic, rotational, or any combination of the three. The transition responsible for luminescence can be stimulated through the release of energy stored in the system chemically or added to the system from an external source. The external source of energy can be of a variety of types including, but not limited to, chemical, thermal, electrical, magnetic, electromagnetic, physical or any other type capable of causing a system to be excited into a state higher than the ground state. For example, a system can be excited by absorbing a photon of light, by being placed in an electrical field, or through a chemical oxidation-reduction reaction. The energy of the photons emitted during luminescence can be in a range from low-energy microwave radiation to high-energy x-ray radiation. Typically, luminescence refers to photons in the range from UV to IR radiation.

The term “read length” as used herein, refers to the number of contiguous nucleic acid residues present in a nucleotide sequence that can be detected or “read” by the methods described herein.

The term “lead” as used herein, refers to an effect of causing a readout of a nucleotide that is at a position that is ahead of or leading the cycle number. For example, when a non-terminated nucleotide is incorporated, then during that same cycle, there is a second opportunity for another nucleotide to be incorporated at the subsequent position in the template strand.

The term “lag” as used herein, refers to an effect of causing a readout in the next cycle that will from the position behind or lagging the cycle number. For example, when no nucleotide is incorporated due for example to polymerase inefficiency, then the site remains available for the next cycle. an effect of causing a readout in the next cycle that will from the position behind or lagging the cycle number.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents exemplary data showing calculated relative predicted versus actual scores when SBS was performed using a Chelation Wash Reagent data was compared to SBS performed with a baseline buffer composition.

FIG. 2 presents exemplary data showing Q score distributions when SBS was performed using a Chelation Wash Reagent data was compared to SBS performed with a baseline buffer composition.

### DETAILED DESCRIPTION OF THE INVENTION

The invention relates to methods, compositions, devices, systems and kits as described including, without limitation, reagents and mixtures for determining the identity of nucleic acids in nucleotide sequences using, for example, sequencing by synthesis (SBS) methods. In particular, the present invention contemplates the use of chelators in washing reagents to improve SBS performance.

### I. Sequencing-By-Synthesis (SBS)

In one embodiment, the present invention contemplates a series of method steps performed by an automated sequencing by synthesis instrument (e.g., a next generation sequencing platform) such as the GeneReader instrument. See U.S. Pat. No. 9,145,589, hereby incorporated by reference. In one embodiment, the instrument is comprised of numerous reagent reservoirs. Each reagent reservoir has a specific reactivity reagent dispensed within the reservoir to support the SBS process, for example:

In one embodiment, the SBS method comprises doing different steps at different stations. By way of example, each station is associated with a particular step. While not limited to particular formulations, some examples for these steps and the associated reagents are shown below:
1) Extend A Reagent: Comprises reversibly terminated labeled nucleotides and polymerase. One composition of Extend A may be as follows:

| Component | Concentration |
|---|---|
| PNSE (% wt/vol) | 0.005% |
| Tris x HCl (pH 8.8), mM | 50 |
| NaCl (mM) | 50 |
| EDTA (mM) | 1 |
| MgSO4 (mM) | 10 |
| Cystamine (mM) | 1 |
| Glycerol (% wt/vol) | 0.01% |
| Therminator IX* (U/ml) | 10 |
| N3-dCTP (μM) | 3.83 |
| N3-dTTP (μM) | 3.61 |
| N3-dATP (μM) | 4.03 |
| N3-dGTP (μM) | 0.4 |
| Alexa488-dCTP (nM) | 550 |
| R6G-dUTP (nM) | 35 |
| ROX-dATP (nM) | 221 |
| Cy5-dGTP (nM) | 66 |

*with Alkylated free Cysteine

2) Extend B Reagent: Comprises reversibly terminated unlabeled nucleotides and polymerase, but lacks labeled nucleotide analogues. One composition of Extend B may be as follows:

| Component | Concentration |
|---|---|
| PNSE (% wt/vol) | 0.005% |
| Tris x HCl (pH 8.8), mM | 50 |
| NaCl (mM) | 50 |
| EDTA (mM) | 1 |
| MgSO4 (mM) | 10 |
| Glycerol (% wt/vol) | 0.01% |
| Thermbator IX* (U/ml) | 10 |
| N3-dCTP (μM) | 21 |
| N3-dTTP (μM) | 17 |
| N3-dATP (μM) | 21 |
| N3-dGTP (μM) | 2 |

*Alkylated free Cysteine

3) Wash solution 1 with a detergent (e.g., polysorbate 20) citrate buffer (e.g., saline)
4) Cleave Reagent: One cleaving solution composition may be as follows:

| Component | Concentration |
|---|---|
| NaOH (mM) | 237.5 |
| TrisHCl (pH 8.0) (mM) | 237.5 |
| TCEP (mM) | 50 |

5) Wash solution 2 with a detergent (e.g., polysorbate 20), a tris(hydroxymethyl)-aminomethane (Tris) buffer and a chelator.

### II. Chelators

A chelator is generally believed to comprise a substance whose molecules can form several bonds to a single metal ion. In other words, a chelator is usually considered to be a multidentate ligand. For example, ethylenediamine:

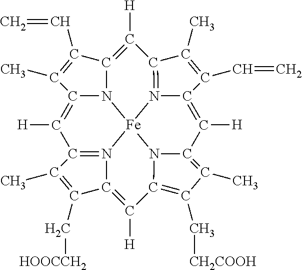

Generally speaking, chelation is a type of bonding of ions and molecules to metal ions. It involves the formation or presence of two or more separate coordinate bonds between a polydentate (multiple bonded) ligand and a single central atom. Usually these ligands are organic compounds, and are called chelants, chelators, chelating agents, or sequestering agents. Chelators are well known for their use in applications such as providing nutritional supplements, in chelation therapy to remove toxic metals from the body, as contrast agents in MRI scanning, in manufacturing using homogeneous catalysts, and in fertilizers.

Numerous biomolecules have been reported to exhibit an ability to dissolve certain metal cations. Thus, proteins, polysaccharides, and polynucleic acids have been demonstrated to act as polydentate ligands for many metal ions. Organic compounds such as the amino acids glutamic acid and histidine, organic diacids such as malate, and polypeptides such as phytochelatin are also typical metal ion chelators. Krämer et al., (1996). “Free histidine as a metal chelator in plants that accumulate nickel”. Nature. 379 (6566): 635-8; Magalhaes, J. V. (2006). “Aluminum tolerance genes are conserved between monocots and divots”. Proceedings of the National Academy of Sciences of the United States of America. 103 (26): 9749-50; and Ha et a., (1999). “Phytochelatin Synthase Genes from Arabidopsis and the Yeast Schizosaccharomyces pombe”. The Plant Cell. 11 (6): 1153-64.

Virtually all metalloenzymes feature metals that are chelated, usually to peptides or cofactors and prosthetic groups. Such chelating agents include the porphyrin rings in hemoglobin and chlorophyll. Many microbial species produce water-soluble pigments that serve as chelating agents, termed siderophores. For example, species of Pseudomonas are known to secrete pyochelin and pyoverdine that bind iron. Enterobactin, produced by E. coli, is the strongest chelating agent known. The marine mussels use metal chelation esp. Fe3+ chelation with the Dopa residues in mussel foot protein-1 to improve the strength of the threads that it uses to secure itself to surfaces. Das et al., (2015). “Tough Coating Proteins: Subtle Sequence Variation Modulates Cohesion”. Biomacromolecules. 16 (3): 1002-8; Harrington et al., (2010). “Iron-Clad Fibers: A Metal-Based Biological Strategy for Hard Flexible Coatings”. Science. 328 (5975): 216-20; and Das et al., (2015). “Peptide Length and Dopa Determine Iron-Mediated Cohesion of Mussel Foot Proteins”. Advanced Functional Materials. 25 (36): 5840-7.

One such known metal chelator is ethylenediaminetetraacetic acid (EDTA), and is believed to be an aminopolycarboxylic acid that is a colorless, water-soluble solid. Its conjugate base is ethylenediaminetetraacetate. EDTA's usefulness arises because of its role as a hexadentate (“six-toothed”) ligand and chelating agent, i.e., its ability to “sequester” metal ions such as Ca2+ and Fe3+. After being bound by EDTA into a metal complex, metal ions remain in solution but exhibit diminished reactivity. EDTA is produced as several salts, notably disodium EDTA and calcium disodium EDTA.

In the laboratory, EDTA is widely used for scavenging metal ions in solutions. Specifically, in biochemistry and molecular biology, ion depletion is commonly used to deactivate metal-dependent enzymes, either as an assay for their reactivity or to suppress damage to DNA or proteins. Dominguez et al. (2009). “A novel nuclease activity that is activated by Ca2+ chelated to EGTA”. Systems Biology in Reproductive Medicine. 55 (5-6): 193-99. In analytical chemistry, EDTA is used in complexometric titrations and analysis of water hardness or as a masking agent to sequester metal ions that would interfere with the analyses. EDTA finds many specialized uses in the biomedical laboratories, such as in veterinary ophthalmology as an anticollagenase to prevent the worsening of corneal ulcers in animals. In tissue culture, EDTA is used as a chelating agent that binds to calcium and prevents joining of cadherins between cells, preventing clumping of cells grown in liquid suspension, or detaching adherent cells for passaging. In histopathology, EDTA can be used as a decalcifying agent making it possible to cut sections using a microtome once the tissue sample is demineralised. EDTA is also known to inhibit a range of metallopeptidases, the method of inhibition occurs via the chelation of the metal ion required for catalytic activity Auld, D. S. (1995). “Removal and replacement of metal ions in metallopeptidases”. Methods In Enzymology. 248: 228-42.

Another well known metal ion chelator ethylene glycol-bis(β-aminoethyl ether)-N,N,N′,N′-tetraacetic acid) (EGTA). EGTA is an aminopolycarboxylic acid and is known to function as a calcium-selective chelating agent and a lower affinity for magnesium as compared to EDTA. Consequently, EGTA is routinely used in physiological buffer solutions homeostatic calcium ions are usually at least a thousand-fold less concentrated than magnesium. Clinically, EGTA has also been used experimentally far the treatment of animals with cerium poisoning and for the separation of thorium from the mineral monazite. EGTA is used as a compound in elution butler in the protein purification technique known as tandem affinity purification, in which recombinant fusion proteins are bound to calmodulin beads and eluted out by adding EGTA. However, there are no reports that EGTA is useful to attach to, and/or sequester, small organic molecules.

However, what is new in the art is an ability of chelators to bind to instrumentation surfaces to prevent binding, displace and/or sequester contaminating compounds.

III. Residual Contaminants from Cleave Reagents

In one embodiment, the present invention contemplates an SBS method comprising a wash reagent comprising at least one chelator. In one embodiment, the wash reagent is used in SBS washing step that follows a cleavage step. Consequently, following the SBS cleave step, contaminate residuals of the compounds comprising a Cleave Reagent may remain in a flow cell (e.g., for example, sodium hydroxide, or tris(hydroxymethyl)-aminomethane HCl, and TCEP). Based on the above discussion, the use of metal dictators would not be expected by one of ordinary skill in the art to bind to, displace or inactivate, any of the compounds in a SBS Cleave Reagent. It was surprising, therefore, to find that a chelator such a EDTA improved SBS performance by addition to a wash reagent that follows the use of a cleave reagent. For example, one compound in a cleave reagent that might be decontaminated by EDTA is TCEP. As a result, one does not necessarily generally consider a chelator to bind to, and or inactive, instrumentation surfaces. For example, instrumentation surfaces that may be contaminated are in synthesis-by-sequencing platforms. It is not well known in the art that EDTA may have an ability to bind to instrumentation surfaces (e.g., SBS instrumentation surfaces) to chelate and/or sequester contaminating compounds.

TCEP (tris(2-carboxyethyl)phosphine) is a reducing agent frequently used in biochemisty and molecular biology applications. Ruegg, U. T & Rudinger, J. (1977). “Reductive cleavage of cysteine disulfides with tributylphosphine”. Methods Enzymol. Methods in Enzymology. 47: 111-116. It is often prepared and used as a hydrochloride salt (TCEP-HCl) with a molecular weight of 286.65 gram/mol having the molecular structure as shown below:

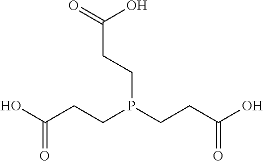 [SMILES: O=C(O)CCP(CCC(=O)O)CCC(=O)O]

TCEP is often used as a reducing agent to break disulfide bonds within and between proteins as a preparatory step for gel electrophoresis. Compared to other common agents used for this purpose including, but not limited to, dithiothreitol and/or β-mercaptoethanol, TCEP has many advantages. Such advantages include, but are not limited to, odorless, a more powerful reducing agent, an irreversible reducing agent (e.g., TCEP does not regenerate—the end product of TCEP-mediated disulfide cleavage is believed to comprise at least two free thiols/cysteines), more hydrophilic, and more resistant to oxidation in air. TCEP also has an advantage in that the compound does not reduce metals. TCEP is soluble in water and available as a stabilized solution at neutral pH and immobilized onto an agarose support to facilitate removal of the reducing agent.

IV. Improved SBS Performance with Wash Reagents Containing a Chelator

SBS sequencing performance (107 cycle sequencing) was compared between runs with a chelation agent in the wash reagent versus runs without a chelation agent in the wash reagent. See Table I.

TABLE 1
SBS Sequencing Performance With/Without Post-Cleave Wash Chelation

| | | | % | % | % @ | | | | |
|---|---|---|---|---|---|---|---|---|---|
| Date | GR | Run Description | mapped | perfect | Q23 | AVG RL | Raw Error | Filtered Error | Output |
| Oct. 21, 2016 | 6.15 | No cleave scavenger In Wash 11 | 19% | 58% | 83% | 68 | 11% | 0.96% | 0.89 Gb |
| Oct. 21, 2016 | 6.8 | No cleave scavenger In Wash 11 | 16% | 57% | 83% | 62 | 14% | 1.19% | 0.56 Gb |
| Oct. 24, 2016 | 6.15 | No cleave scavenger In Wash 11 | 27% | 61% | 86% | 68 | 10% | 0.85% | 0.53 Gb |
| Oct. 4, 2016 | 8.17 | EDTA 25 mM In Wash 11 | 29% | 61% | 86% | 92 | 2.60% | 0.69% | 1.1 Gb |
| Oct. 13, 2016 | 8.31 | EDTA 50 mM + 3X Vol | 8.31 | 35% | 87% | 92 | 2.67% | 0.62% | 1.2 Gb |
| Oct. 13, 2016 | 8.17 | EDTA 50 mM + 3X Vol | 8.17 | 37% | 87% | 92 | 2.61% | 0.63% | 1.2 Gb |

The data show significantly improvements SBS performance as shown by longer read lengths and reduced error rates when a chelation agent (e.g., EDTA) was present in a post-cleavage washing step. The data was verified under the conditions where cystamine, normally present in a Wash Reagent, was removed and replaced with an EDTA Chelation Wash Reagent comprising new nucleotides. See, Table II.

TABLE II
SBS Sequencing Performance When EDTA Replaces Cystamine In Wash Reagent

| Run Date | Run Description | GR | % Mapped | % Perfect | % @ Q25 | AVG RL | Error (FQ) | Error (FASTQ) | Output (Gb) |
|---|---|---|---|---|---|---|---|---|---|
| Sep. 23, 2016 | Baseline | 5.19 | 31% | 55% | 86% | 94.22 | 2.41% | 0.66% | 1.10 |
| Oct. 4, 2016 | EDTA 25 mM | 8.17 | 29% | 61% | 86% | 91.58 | 2.27% | 0.69% | 1.00 |
| Oct. 9, 2016 | EDTA 25 mM | 5.19 | 31% | 61% | 85% | 89.25 | 2.52% | 0.69% | 0.94 |
| Oct. 9, 2016 | EDTA 25 mM | 8.17 | 29% | 56% | 84% | 83.10 | 4.15% | 0.85% | 0.96 |

Even though the data shows some raw error rate variability, one of skill in the art would likely attribute this variability to ordinary lot-to-lot differences resulting from instrumentation settings and/or nucleotide mixes. It is believed that these results are reproducible at higher guanosine diphosphate (GDP) levels.

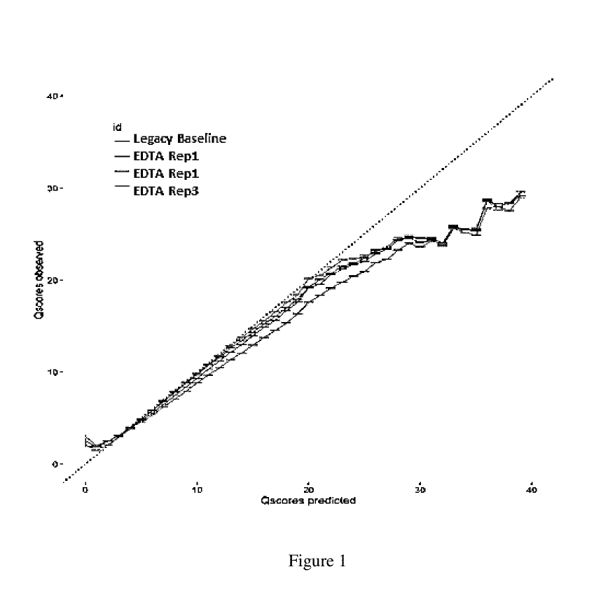

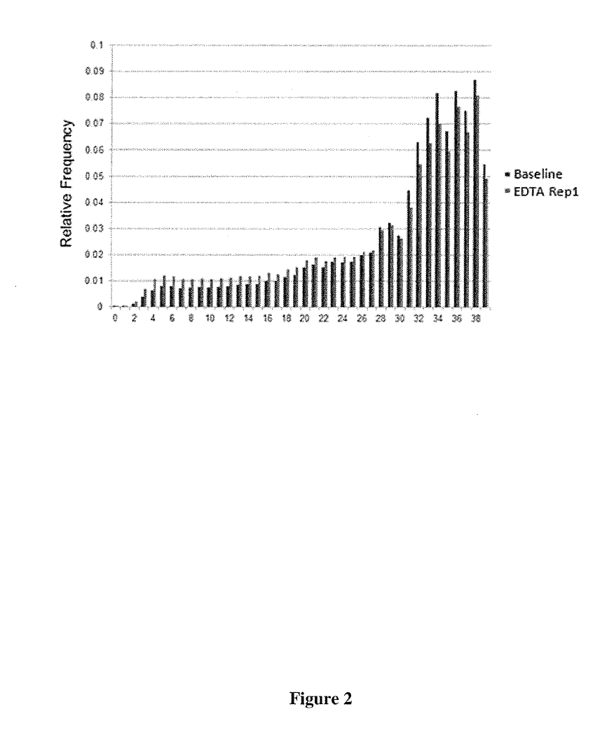

When SBS performed using a Chelation Wash Reagent data was compared to SBS performed with a baseline buffer composition, the data showed the calculated relative Q-scores were very similar. See, FIG. 1. The Q-Score distribution pattern obtained with a Chelation Wash Reagent was also comparable to that performed with a baseline SBS buffer and demonstrated well defined peaks at high Q-score values. See, FIG. 2.

The Chelation Wash Reagent was also shown to retain comparable performance to baseline SBS buffers even when using a known genomic hotspot region (e.g., V2 101x-GR) Current configuration (EDTA) as compared to a standard quality-based CLC variant analysis. See, Table III.

TABLE III
Chelation Wash Reagent Comparison Using V2 101x-GR Hotspot 1.5% AF
Acrometrix

| | AVG | >500x | | | | | | | Average |
|---|---|---|---|---|---|---|---|---|---|
| SBS | Read | Read | | | | All | All | All | Error Rate |
| Configuration Type | Length | Coverage | All TPs | All FPs | All FNs | Sensitivity | Specificity | Precision | (%) |
| NewSBS/EDTA | 90.23 | 100 | 73 | 0 | 0 | 1 | 1 | 1 | 0.53 |
| 25 mM Condition | | | | | | | | | |
| (AddA) | | | | | | | | | |
| NewSBS/EDTA | 90.03 | 100 | 73 | 0 | 0 | 1 | 1 | 1 | 0.54 |
| 25 mM Condition | | | | | | | | | |
| (AddA) | | | | | | | | | |
| NewSBS/EDTA | 90.96 | 100 | 73 | 0 | 0 | 1 | 1 | 1 | 0.5 |
| 25 mM Condition | | | | | | | | | |
| (AddA) | | | | | | | | | |
| NewSBS/EDTA | 92.85 | 100 | 73 | 0 | 0 | 1 | 1 | 1 | 0.45 |
| 50 mM Condition | | | | | | | | | |
| (AddA) | | | | | | | | | |
| NewSBS/EDTA | 92.57 | 100 | 73 | 0 | 0 | 1 | 1 | 1 | 0.47 |
| 50 mM Condition | | | | | | | | | |
| (AddA) | | | | | | | | | |
| NewSBS/EDTA | 93.45 | 100 | 73 | 0 | 0 | 1 | 1 | 1 | 0.45 |
| 50 mM Condition | | | | | | | | | |
| (AddA) | | | | | | | | | |
| NewSBS/EDTA | 93.38 | 100 | 73 | 0 | 0 | 1 | 1 | 1 | 0.47 |
| 50 mM Condition | | | | | | | | | |
| (AddA) | | | | | | | | | |
| GR1.0 Product | 81.57 ± 1.57 | 0.99 ± 0.01 | 57.75 ± 0.45 | 1.25 ± 0.46 | 1.00 ± 1.20 | 0.98 ± 0.01 | 1.00 ± 0.01 | 0.99 ± 0.02 | 0.50 ± 0.03 |
| Configuration | | | | | | | | | |
| SY2 (AddC) | | | | | | | | | |
| GR1.0 Hybrid | 98.19 | 100 | 73 | 0 | 0 | 1 | 1 | 1 | 0.49 |
| Configuration | | | | | | | | | |
| (AddA) | | | | | | | | | |
| GR1.0 Hybrid | 98.16 | 100 | 73 | 0 | 0 | 1 | 1 | 1 | 0.5 |
| Configuration | | | | | | | | | |
| (AddA) | | | | | | | | | |

AddA = Indole Proprionic Acid;
AddC = Vitamin C

These data show that the Chelation Wash Reagent provided a lower error rate and better performance on TPs, FPs and FNs in comparison with a commercially available GR1.0 Product Configuration.

Preliminary SBS sequencing results using clinical sample pools (e.g., AMP 2016; 8-plex samples) demonstrated that the Chelation Wash Reagent performed as an equivalent when compared to two known baseline SBS methods (e.g., SV2 GR1.0 and GR1.1). See, Table IV.

TABLE IV
CLINICAL SAMPLE EQUIVALENCY TO KNOWN BASELINE SBS METHODS

| | | | | | | PRODUCT KPI's | | | | | |
|---|---|---|---|---|---|---|---|---|---|---|---|
| | | RUN VALIDITY CRITERIA | | | | | | ERROR | | PERFORMANCE | |
| | CLINICAL | BEAD/ | % BEAD | | % | % @ | AVG | RATE | OUTPUT | METRICS | |
| AMP 2016 | POOL | TILE | RETENTION | % LIVE | MAPPED | Q25 | RL | (MFST) | (Gb) | LEAD | LAG |
| | DH4 | 436k | 97% | 53% | 30% | 87% | 92 | 0.48% | 1.08 | 0.6 | 0.07 |
| | DH3 | 446k | 97% | 46% | 30% | 85% | 86 | 0.55% | 0.91 | 0.62 | 0.07 |
| | DH7 | 428k | 97% | 50% | 30% | 86% | 90 | 0.51% | 1.08 | 0.56 | 0.14 |
| | DH6 | 418k | 97% | 49% | 30% | 87% | 91 | 0.49% | 1.03 | 0.6 | 0.08 |
| | DH5 | 452k | 97% | 51% | 32% | 85% | 90 | 0.48% | 1.1 | 0.58 | 0.08 |
| | DH8 | 433k | 97% | 50% | 29% | 86% | 87 | 0.51% | 0.97 | 0.59 | 0.07 |
| | DH2 | 426k | 97% | 49% | 29% | 85% | 87 | 0.49% | 1.03 | 0.54 | 0.08 |
| | DH1 | 427k | 97% | 48% | 28% | 86% | 85 | 0.48% | 0.98 | 0.6 | 0.07 |
| AVERAGE | | 433k | 97% | 50% | 30% | 85.6% | 88.4 | 0.50% | 1.02 | 0.59 | 0.08 |
| Adev | | 12345 | 0.50% | 4% | 3% | 1% | 5.3 | 0.03% | 0.06 | 0.06 | 0.06 |
| | | | | | | | | ERROR | | | |
| | | BEAD/ | % BEAD | | % | % @ | AVG | RATE | OUTPUT | | |
| SV2 GR1.0 | n = 70 | TILE | RETENTION | % LIVE | MAPPED | Q25 | RL | (MFST) | (Gb) | LEAD | LAG |
| | AVERAGE | 419k | 97.9% | 54.2% | 35.9% | 85.8% | 79.5 | 0.49% | 1.08 | 0.36 | 0.12 |
| | SDev | 10438 | 0.5% | 4.6% | 3.3% | 0.7% | 1.4 | 0.03% | 0.10 | 0.03 | 0.02 |
| | | | | | | | | ERROR | | | |
| | | BEAD/ | % BEAD | | % | % @ | AVG | RATE | OUTPUT | | |
| GR1.1 | n = 2 | TILE | RETENTION | % LIVE | MAPPED | Q25 | RL | (MFST) | (Gb) | LEAD | LAG |
| | AVERAGE | 430k | 96.9% | 52.2% | 35.9% | 86.2% | 95.0 | 0.49% | 1.10 | 0.26 | 0.13 |
| | SDev | 11432 | 0.1% | 1.6% | 1.3% | 0.5% | 1.1 | 0.05% | 0.10 | 0.05 | 0.03 |

The completed analysis for AMP 2016 clinical pools revealed 13 RAS positives and 29 WT's. Furthermore, the above suggested equivalency for major KPIs to GRIM performance/legacy chemistry was confirmed. Table V.

TABLE V
COMPLETE AMP CLINICAL SAMPLE SUMMARY

| | | | | | | PRODUCT KPI's | | | | | |
|---|---|---|---|---|---|---|---|---|---|---|---|
| | | RUN VALIDITY CRITERIA | | | | | | ERROR | | PERFORMANCE | |
| | | BEAD/ | % BEAD | | % | % @ | AVG | RATE | OUTPUT | METRICS | |
| AMP 2016 | n(runs) = 11 | TILE | RETENTION | % LIVE | MAPPED | Q25 | RL | (MFST) | (Gb) | LEAD | LAG |
| | DH4 | 436k | 97% | 53% | 30% | 87% | 92 | 0.48% | 1.06 | 0.6 | 0.07 |
| | DH3 | 446k | 97% | 46% | 30% | 85% | 86 | 0.55% | 0.91 | 0.62 | 0.07 |
| | DH7 | 428k | 97% | 50% | 30% | 86% | 90 | 0.51% | 1.08 | 0.56 | 0,14 |
| | DH6 | 418k | 97% | 49% | 30% | 87% | 91 | 0.49% | 1.03 | 0.6 | 0.08 |
| | DH5 | 452k | 97% | 51% | 32% | 85% | 90 | 0.48% | 1.1 | 0.58 | 0.08 |
| | DH8 | 433k | 97% | 50% | 29% | 86% | 87 | 0.51% | 0.97 | 0.59 | 0.07 |
| | DH2 | 426k | 97% | 49% | 29% | 85% | 87 | 0.49% | 1.03 | 0.54 | 0.08 |
| | DH1 | 427k | 97% | 48% | 28% | 86% | 85 | 0.48% | 0.98 | 0.6 | 0.07 |
| | DH9 | 422k | 96% | 48% | 32% | 86% | 90 | 0.58% | 1.05 | 0.59 | 0.07 |
| | DH10 | 423k | 97% | 46% | 34% | 87% | 92 | 0.51% | 1.17 | 0.54 | 0.08 |
| | DH11 | 425k | 97% | 54% | 38% | 85% | 85 | 0.58% | 1.15 | 0.6 | 0.07 |
| AVERAGE | | 433k | 97% | 50% | 31% | 85.7% | 88.8* | 0.50% | 1.05 | 0.59 | 0.08 |
| ADev | | 12345 | 0.50% | 4% | 3% | 1% | 5.3 | 0.03% | 0.06 | 0.06 | 0.06 |
| | | | | | | | | ERROR | | | |
| | | BEAD/ | % BEAD | | % | % @ | AVG | RATE | OUTPUT | | |
| SV2 GR1.0 | n(runs) = 70 | TILE | RETENTION | % LIVE | MAPPED | Q25 | RL | (MFST) | (Gb) | LEAD | LAG |
| | AVERAGE | 419k | 97.9% | 54.2% | 35.9% | 85.8% | 79.5 | 0.49% | 1.08 | 0.36 | 0.12 |
| | SDev | 10438 | 0.5% | 4.6% | 3.3% | 0.7% | 1.4 | 0.03% | 0.10 | 0.03 | 0.02 |
| | | | | | | | | ERROR | | | |
| | | BEAD/ | % BEAD | | % | % @ | AVG | RATE | OUTPUT | | |
| GR1.1 | n(runs) = 2 | TILE | RETENTION | % LIVE | MAPPED | Q25 | RL | (MFST) | (Gb) | LEAD | LAG |
| | AVERAGE | 430k | 96.9% | 52.2% | 38.9% | 86.2% | 95.0 | 0.49% | 1.10 | 0.26 | 0.13 |
| | SDev | 11432 | 0.1% | 1.6% | 1.3% | 0.5% | 1.1 | 0.05% | 0.10 | 0.05 | 0.03 |

It should be noted that an increased ARL was favored by Additive A (ascorbic acid), which is not present in the GR 1.0 baseline runs (SV2 1.0).

Furthermore, the Chelation Wash Reagent was observed to be effective regardless of the SBS instrumentation platform as shown by concordance data. See, Table VI.

TABLE VI
SBS Instrument Concordance Using Chelation Wash Reagents

| | | RAS Variant Allele Frequency (%) | | | |
|---|---|---|---|---|---|
| | KRAS | TherascreenPCR/ | GeneReader/ | GeneReader/ | |
| Sample # | AA Change | Pyro | New Chemistry | Legacy Chemistry | MiSeq |
| 1 | A146P | 41% | 31% | 40% | 32% |
| 2 | Q61H | 36% | 41% | 35% | 26% |
| 3 | Q61H | 31% | 25% | 22% | 35% |
| 5 | G12D | + | 44% | 45% | 11% |

For example, these preliminary data show a 100% positive agreement between the GeneReader and MiSeq SBS instrument platforms.

The observed concordance between these two instrument platforms was then confirmed with additional sequencing runs. See, Table VII.

TABLE VII
Instrument Concordance Between SBS Instrument Platforms

| | | RAS Variant Allele Frequency (%) | | | |
|---|---|---|---|---|---|
| | KRAS | TherascreenPCR/ | GeneReader/ | GeneReader/ | |
| Sample # | AA Change | Pyro | New Chemistry | Legacy Chemistry | MiSeq |
| 1 | G12D | + | 20 | 21 | 7 |
| 2 | G12D | + | 37 | 39 | 12 |
| 3 | A59T | 19 | 16 | 15 | 14 |
| 4 | G12D | + | 44 | 45 | 11 |
| 5 | Q61H | 14 | 10 | 9 | 13 |
| 8 | A146P | 41 | 42 | 40 | 32 |
| 7 | Q61H | 36 | 41 | 35 | 26 |
| 3 | Q61H | 32 | 25 | 22 | 35 |
| 9 | K117N | 24 | 36 | 34 | 39 |
| 10 | G13D | + | 44 | 47 | 15 |
| 11 | G12C | + | 32 | 32 | 10 |
| 12 | G13D | + | 27 | 34 | 10 |
| 13 | Q61H | 26 | 26 | 21 | 23 |

+: Mutant identified by therascreen RGQ; allele frequency not available

These data show a 100% positive agreement between the GeneReader and MiSeq SBS instrument platforms. This concordance was also seen with more conventional sequencing methods (e.g., Therascreen PCR and Pyro assays). See, Table VIII.

TABLE VII
GeneReader NGS System Concordance with PCR and Pyro Assays

| RAS agreement study | | | | |
|---|---|---|---|---|
| >5% Ras variant Allele | | Therascreen PCR and Pyro Assays⁽¹⁾ | | |
| Frequency Cutoff | | + (MT) | − (WT) | Total |
| GeneReader | + (MT) | 13 | 0 | 13 |
| NGS | − (WT) | 0 | 29 | 29 |
| System⁽²⁾ | Total | 13 | 29 | 42 |

⁽¹⁾If KRAS status regarded as mutant (MT) as identified by either therascreen KRAS PCR Assay or by therascreen RAS Extension Pyro Assay.
⁽²⁾Variants from codons 12, 13, 59, 61, 117, 146 contained in established QIAGEN therascreen assays are called.

These data show a 100% Agreement between GeneReader NGS System and QIAGEN Therascreen PCR & Pyro Assays.

We claim:

1. A method of incorporating labeled nucleotides, comprising:
a) providing;
i) a plurality of nucleic acid primers and template molecules,
ii) a polymerase,
iii) a washing reagent comprising a chelator, and
iv) a plurality of nucleotide analogues wherein at least a portion of said nucleotide analogues is labeled with a label attached through a cleavable linker to the base;
b) hybridizing at least a portion of said primers to at least a portion of said template molecules so as to create hybridized primers;
c) incorporating a first labeled nucleotide analogue with said polymerase into at least a portion of said hybridized primers so as to create extended primers comprising an incorporated labeled nucleotide analogue;
d) cleaving said extended primers comprising an incorporated labeled nucleotide analogue with a solution comprising a cleave reagent; and
e) washing said incorporated labeled nucleotide analogue in the presence of said washing reagent.

2. The method of claim 1, wherein said cleave reagent is selected from the group consisting of tris(2-carboxyethyl)phosphine and tris(hydroxymethyl)-aminomethane HCl.

3. The method of claim 1, wherein said incorporating is performed within a flow cell.

4. The method of claim 1, wherein said cleave reagent compound contacts a metal surface.

5. The method of claim 1, wherein said chelator contacts a metal surface.

6. The method of claim 5, wherein said metal surface comprises an iron-containing surface.

7. The method of claim 6, wherein said iron-containing surface is selected from the group consisting of an iron silicate surface, a ferrotitanium surface and a magnetic bead ferrite surface.

8. The method of claim 1, wherein said chelator is selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt.

9. The method of claim 1, further comprising step (f) incorporating additional nucleotide analogues with said polymerase into at least a portion of said extended primers.

10. The method of claim 9, wherein said additional nucleotide analogues are incorporated into said at least a portion of said extended primer with a reduced error rate as compared to the error rate with a wash reagent without a chelator.

11. The method of claim 9, wherein said at least a portion of said extended primer comprises a longer read length as compared to a wash reagent without a chelator.

12. The method of claim 1, wherein said label is fluorescent.

13. A washing reagent comprising at least one chelator and a buffer.

14. The washing reagent of claim 13, wherein said at least one chelator comprises a compound selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt.

15. The washing reagent of claim 13, wherein said reagent further comprises a detergent.

16. The washing reagent of claim 15, wherein said detergent is a polysorbate.

17. The washing reagent of claim 13, wherein said buffer is a TRIS buffer.

18. The washing reagent of claim 13, wherein said buffer is a HEPES buffer.

19. A kit, comprising:
i) a first container comprising a washing reagent comprising at least one chelator and a buffer; and
ii) a second container comprising a plurality of nucleotide analogues wherein at least a portion of said nucleotide analogues is labeled with a label attached through a cleavable linker to the base.

20. The kit of claim 19, wherein said at least one chelator comprises a compound selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt.

21. The kit of claim 19, wherein said reagent further comprises a detergent.

22. The kit of claim 21, wherein said detergent is a polysorbate.

23. The kit of claim 19, wherein said buffer is a TRIS buffer.

24. The kit of claim 19, wherein said buffer is a HEPES buffer.

25. A system comprising a solution of primers hybridized to a template comprising a plurality of nucleotide analogues attached to a cleavable label and a washing reagent comprising at least one chelator and a buffer.

26. The system of claim 25, wherein said hybridized primers and said template are immobilized.

27. The system of claim 25, wherein said hybridized primers and said template are in a flow cell.

28. The system of claim 25, wherein said at least one chelator comprises compounds selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt.

29. The system of claim 25, wherein said reagent further comprises at least one detergent.

30. The system of claim 29, wherein said at least one detergent is a polysorbate.

31. The system of claim 25, wherein said buffer is a TRIS buffer.

32. The system of claim 25, wherein said buffer is a HEPES buffer.

33. A mixture comprising a solution of primers hybridized to a template comprising a plurality of nucleotide analogues attached to a cleavable label and a washing reagent comprising at least one chelator and a buffer.

34. The mixture of claim 33, wherein said hybridized primers and said template are immobilized.

35. The mixture of claim 33, wherein said hybridized primers and said template are in a flow cell.

36. The mixture of claim 33, wherein said at least one chelator comprises compounds selected from the group consisting of ethylenediaminetetraacetic acid, ethylene glycol-bis(β-aminoethyl ether)-N,N,N′,N′-tetraacetic acid, iminodisuccinic acid, polyaspartic acid, ethylenediamine-N,N′-disuccinic acid, methylglycinediacetic acid, and/or L-glutamic acid N,N-diacetic acid, tetrasodium salt.

37. The mixture of claim 33, wherein said reagent further comprises at least one detergent.

38. The mixture of claim 37, wherein said at least one detergent is a polysorbate.

39. The mixture of claim 33, wherein said buffer is a TRIS buffer.

40. The mixture of claim 33, wherein said buffer is a HEPES buffer.

41. A washing reagent comprising:
i) a TRIS HCl buffer;
ii) polysorbate 20; and
iii) ethylenediaminetetraacetic acid ranging in concentration between approximately 25-50 mM.
